(19) United States
(12) Patent Application Publication
(10) Pub. No.: US 2025/0193536 A1
(43) Pub. Date: Jun. 12, 2025

(54) THREE-DIMENSIONAL OPTICAL IMAGING SYSTEM AND METHOD

(71) Applicant: HEFEI RAYCISION MEDICAL TECHNOLOGY CO., LTD., Hefei (CN)

(72) Inventor: Yirui YANG, Hefei (CN)

(21) Appl. No.: 18/966,172

(22) Filed: Dec. 3, 2024

Related U.S. Application Data
(63) Continuation of application No. PCT/CN2024/089577, filed on Apr. 24, 2024.

(30) Foreign Application Priority Data
Dec. 7, 2023 (CN) 202311668083.7

(51) Int. Cl. H04N 23/95; G01B 11/25; G06T 7/33; G06T 7/80

(52) U.S. Cl. CPC H04N 23/95; G01B 11/2513; G06T 7/33; G06T 7/80; G06T 2207/10064; G06T 2207/20212

(57) ABSTRACT

Provided are a 3D optical imaging system and method. The method includes the following steps: S1, obtaining a two-dimensional bio-optical image of an imaging target by a CCD camera; S2, obtaining a three-dimensional surface contour image of the imaging target by using the CCD camera in step S1 in combination with structured light; S3, performing an alignment on the two-dimensional bio-optical image obtained in step S1 and the three-dimensional surface contour image obtained in step S2; and S4, reconstructing a three-dimensional bioluminescence image.

### CROSS-REFERENCE TO RELATED APPLICATION

The present application is a continuation of International Patent Application No. PCT/CN2024/089577, filed on Apr. 24, 2024, which claims priority to Chinese patent application No. 202311668083.7, titled “THREE-DIMENSIONAL OPTICAL IMAGING SYSTEM AND METHOD” and filed on Dec. 7, 2023, the entire contents of which are incorporated herein by reference.

### FIELD

The present disclosure relates to the field of life sciences and medical imaging device technologies, and more particularly, to a 3D optical imaging system and a 3D optical imaging method.

### BACKGROUND

Bio-optical imaging is widely used due to its advantages of mature development of detection instruments, high sensitivity, high contrast, high resolution, intuitive imaging, fast imaging speed, and non-invasive detection. It has important practical significance and application prospects in exploring pathogenesis, clinical manifestations, genetic lesions of diseases, understanding corresponding physiological and pathological information, diagnosing diseases, developing new medical treatments, and the like.

The bio-optical imaging refers to imaging molecules, cells, tissues, organisms, or the like using optical detection means in combination with chemiluminescence or an excitation fluorescence mechanism, which is an important method to obtain biological information. Depending on different detection methods, the bio-optical imaging can be categorized into molecular fluorescence imaging, bioluminescence imaging, photoacoustic imaging, optical tomography imaging, and so on.

Structured light imaging projects specific structured light (e.g., stripes, lattices, etc.) onto a surface of a capturing object, and then records deformations of the structured light on the surface of the object using a camera to derive 3D shape information of the surface of the object. The structured light imaging typically is composed of three main components: a projection system, a camera, and a computer processing system. The projection system usually illuminates a to-be-measured object through projecting a grating or a stripe pattern. A shape and a size of the grating or the stripe pattern can be adjusted as desired. The camera is used to record a structured light pattern on the surface of the to-be-measured object and convert the structured light pattern into a digital image. A resolution and a collection speed of the camera have a great impact on imaging precision and real-time performance. The computer processing system is used to process collected image data and reconstruct a 3D shape of the to-be-measured object based on information about the deformations of the structured light. The process usually includes steps such as image preprocessing, camera calibration, 3D reconstruction, and data visualization. With advantages of non-contact, high precision, and high efficiency, the structured light imaging technique is widely applied in industrial manufacturing, medical imaging, cultural relics protection, virtual reality, and other fields.

In conventional bio-optical imaging systems, the bio-optical imaging is used to image cells or tissues or even an organism to obtain biological information therein. However, the conventional bio-optical imaging systems are mainly confined to two-dimensional surface imaging, making it difficult to obtain a three-dimensional distribution of bio-optical signals in the organism.

Therefore, a 3D optical imaging system and a 3D optical imaging method are urgently needed. Compared with the related art, the 3D optical imaging system and the 3D optical imaging method obtain a three-dimensional spatial distribution of bio-optical signals in the organism.

### SUMMARY

The present disclosure solves a technical problem in the related art, and provides a 3D optical imaging system and a 3D optical imaging system method.

To realize the above objective, the present disclosure adopts the following technical solutions.

A 3D optical imaging method includes the following steps: S1, obtaining a two-dimensional bio-optical image of an imaging target by a CCD camera, the two-dimensional bio-optical image being a bioluminescence image or a molecular fluorescence image; S2, obtaining a three-dimensional surface contour image of the imaging target by using the CCD camera in step S1 in combination with structured light; S3, performing an alignment on the two-dimensional bio-optical image obtained in step S1 and the three-dimensional surface contour image obtained in step S2; and S4, obtaining a three-dimensional solid structure through filling the obtained three-dimensional surface contour image with biological tissues having different optical properties, and reconstructing a three-dimensional bioluminescence image or a three-dimensional molecular fluorescence image through combining the three-dimensional solid structure with data obtained based on the alignment in step S3. Further, step S2 includes the following steps: S201, turning on a projector, projecting modulated stripe-patterned structured light onto a surface of the imaging target, and capturing stripes on the surface of the imaging target using the CCD camera; S202, processing the stripes to obtain a phase distribution map of the surface of the imaging target; S203, obtaining a phase-coordinate relationship subsequent to a geometric calibration, and converting the phase distribution into three-dimensional coordinates using the phase-coordinate relationship obtained subsequent to the geometric calibration; S204, adjusting an angle between an imaging support and an imaging system, and repeating steps S201 to S203; and S205, obtaining multi-angle three-dimensional coordinates of the imaging target by adjusting the angle, such that the three-dimensional surface contour image of the imaging target is obtained.

Further, the phase distribution map of the surface of the imaging target is obtained through: obtaining stripe images of different phases contained in images captured each time, obtaining a wrapped phase distribution of the stripes through performing an algebraic operation and a stitching operation on the stripe images, and performing a spatial phase expansion on wrapped phases based on spatial sequence information of the stripes to obtain the phase distribution map of the surface of the imaging target.

Further, the geometric calibration of the phase-coordinate relationship is performed by: given phases and CCD camera image coordinates that are known, converting the CCD camera image coordinates into three-dimensional coordinates in a CCD camera coordinate system using a camera parameter, converting the three-dimensional coordinates in the CCD camera coordinate system into three-dimensional coordinates in a projector coordinate system based on a relative parameter between the projector and the CCD camera, converting the three-dimensional coordinates in the projector coordinate system into projector image coordinates based on a projector parameter, and obtaining the phase-coordinate relationship based on a one-to-one correspondence between the phases and the projector image coordinates.

Further, the molecular fluorescence image is obtained through: turning on an excitation light source, emitting laser light by the excitation light source to irradiate the imaging target, exciting fluorescent molecules carried by an imaging object, and generating emission fluorescence; and obtaining the two-dimensional bio-optical image through collecting and processing, by the CCD camera, the generated emission fluorescence subsequent to the generated emission fluorescence being reflected by a reflection mirror and passing through a filter, or subsequent to the generated emission fluorescence passing through the filter without being reflected.

Further, the bioluminescence image is obtained through: releasing a bioluminescence signal in response to a chemical reaction inside an imaging object, and obtaining a two-dimensional biological image through collecting and processing, by the CCD camera, the released bioluminescence signal subsequent to the released bioluminescence signal being reflected by a reflection mirror and passing through a filter, or obtaining the two-dimensional bio-optical image through collecting and processing, by the CCD camera, the released bioluminescence signal subsequent to the released bioluminescence signal passing through the filter without being reflected, or obtaining the two-dimensional bio-optical image through directly collecting and processing, by the CCD camera, the released bioluminescence signal without the released bioluminescence signal being reflected or passing through the filter.

Further, the data obtained based on the alignment in step S3 described in step S4 includes a correspondence between points on the two-dimensional bio-optical image and points on the three-dimensional surface contour image and a corresponding optical signal intensity.

A 3D optical imaging system is provided, which uses the 3D optical imaging method described above. The 3D optical imaging system includes the imaging support and the imaging system. The imaging system includes the excitation light source and the CCD camera. The imaging target is fixed at the imaging support. The excitation light source and the CCD camera are disposed at a same side of the imaging target or the excitation light source and the CCD camera are disposed at two sides of the imaging target respectively. The projector is disposed at a side of the imaging target.

Further, the reflection mirror is disposed between the imaging target and the CCD camera. The projector is disposed at a side of the reflection mirror.

Further, the filter is disposed between the reflection mirror and the CCD camera.

Compared with the related art, the present disclosure can provide the following advantageous effects. The two-dimensional bio-optical image and the three-dimensional surface contour image of the imaging target are obtained using a same CCD camera. The alignment is performed on the two-dimensional bio-optical image and the three-dimensional surface contour image. The three-dimensional bioluminescence image can be obtained through filling the three-dimensional surface contour image with biological tissues having different optical properties and in combination with data obtained subsequent to the alignment. In this way, a three-dimensional spatial distribution of bio-optical signals in an organism is obtained.

### BRIEF DESCRIPTION OF THE DRAWINGS

FIG. 1 is a structural diagram of a system according to the present disclosure.

FIG. 2 is a flowchart of a method according to the present disclosure.

### DETAILED DESCRIPTION

Technical solutions of the present disclosure will be described clearly below in combination with accompanying drawings. Obviously, the embodiments described below are not all embodiments of the present disclosure. All other embodiments obtained by those skilled in the art without creative labor shall fall within the protection scope of the present disclosure.

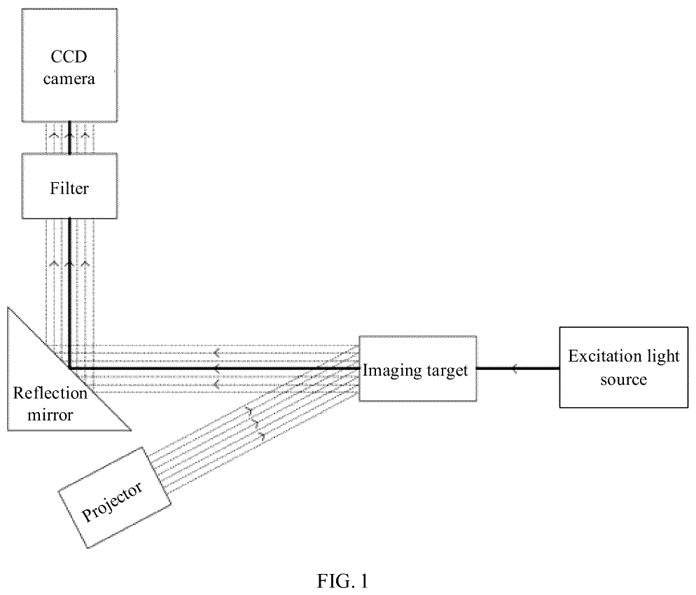

As illustrated in FIG. 1, the present disclosure provides a 3D optical imaging system including an imaging support and an imaging system. The imaging system includes an excitation light source and a CCD camera. The imaging support is realized as a carrier table or an imaging bed and is configured to fix an imaging target. The imaging target may be fixed at the imaging support in a vertical direction or a horizontal direction. The excitation light source and the CCD camera are disposed at a same side of the imaging support or are disposed at two sides of the imaging support respectively. The excitation light source may be a laser source, a LED light source, a halogen light source, etc. A type of the excitation light source is not specifically limited in the present disclosure.

In this embodiment, the excitation light source is disposed at a right side of the imaging target. Further, a reflection mirror is disposed between the imaging target and the CCD camera (or the CCD camera may be directly used for a collection without providing a reflection mirror). The excitation light source, the imaging target, and the reflection mirror are sequentially arranged in a direction of illumination of the excitation light source. The CCD camera is disposed above the reflection mirror. A filter is disposed between the CCD camera and the reflection mirror.

Further, a projector is disposed at a side of the reflection mirror. An angle between the imaging support and the imaging system can be rotated and adjusted. Each of the excitation light source and the projector is aligned with the imaging target fixed at the imaging support. Light emitted by the excitation light source and the projector is irradiated on the imaging target. Fluorescent light excited by the excitation light source and emitted light from the projector reflected by the imaging object are reflected by the reflection mirror and then collected by the CCD camera.

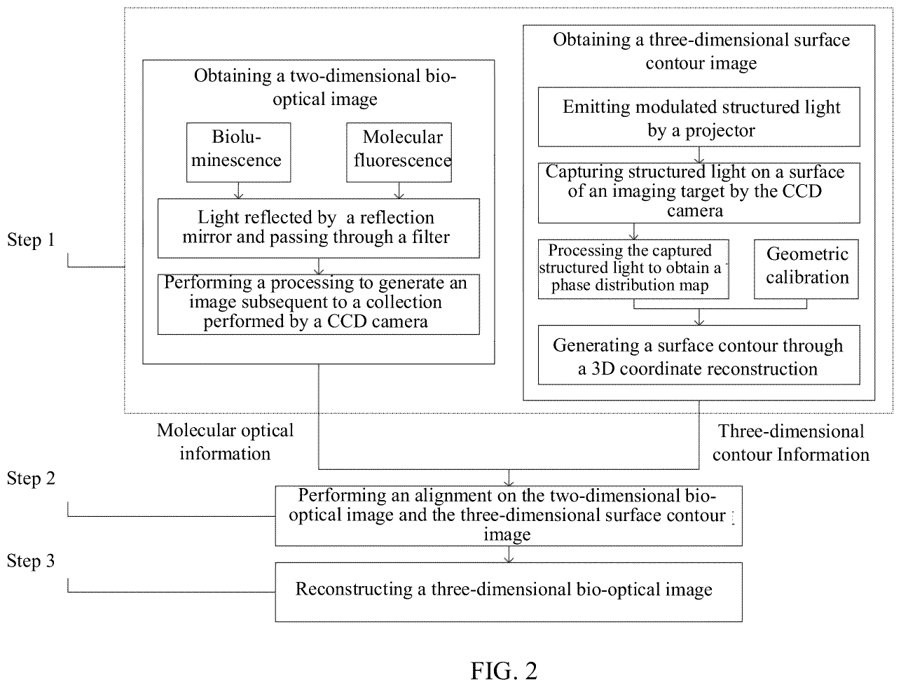

As illustrated in FIG. 2, the present disclosure further provides a 3D optical imaging method. The 3D optical imaging method includes the following steps.

In S1, a two-dimensional bio-optical image of an imaging target is obtained. The two-dimensional bio-optical image includes images of two modalities, a bioluminescence image and a molecular fluorescence image. In this step, only one of the bioluminescence image and the molecular fluorescence image needs to be obtained.

Specifically, the bioluminescence image is obtained through: releasing a bioluminescence signal in response to a chemical reaction inside the imaging object, and obtaining a two-dimensional biological image through collecting and processing, by the CCD camera, the released bioluminescence signal subsequent to the released bioluminescence signal being reflected by a reflection mirror and passing through a filter, or obtaining the two-dimensional bio-optical image through collecting and processing, by the CCD camera, the released bioluminescence signal subsequent to the released bioluminescence signal passing through the filter without being reflected, or obtaining the two-dimensional bio-optical image through directly collecting and processing, by the CCD camera, the released bioluminescence signal without the released bioluminescence signal being reflected or passing through the filter.

The chemical reaction inside the imaging object comes from an enzymatic reaction in an organism, which is autofluorescence in an animal body. An enzyme that catalyzes such a reaction is called luciferases. A common method is to construct an expression vector for luciferase genes, transfect the luciferase genes into target cells, and then transplant the target cells into a target organ of a recipient. During an observation, exogenous luciferin is injected into the target cells. In this way, a reaction occurs within the target cells, producing fluorescence. Then, real-time monitoring of an expression of the target cells or target molecules can be implemented using a living biological optical imaging system with high-sensitivity.

The molecular fluorescence image is obtained through: turning on an excitation light source, emitting laser light by the excitation light source to irradiate the imaging target, exciting fluorescent molecules carried by an imaging object, and generating emission fluorescence; and obtaining the two-dimensional bio-optical image through collecting and processing, by the CCD camera, the generated emission fluorescence subsequent to the generated emission fluorescence being reflected by a reflection mirror and passing through a filter, or subsequent to the generated emission fluorescence passing through the filter without being reflected.

In S2, a three-dimensional surface contour image of the imaging target is obtained. Specifically, step S2 includes the following steps.

In S201, a projector is turned on, modulated stripe-patterned structured light is projected onto a surface of the imaging target, and stripes on the surface of the imaging target are captured using the CCD camera.

In S202, the stripes are processed, and the phase distribution map of the surface of the imaging target is obtained through: obtaining stripe images of different phases contained in images captured each time, obtaining a wrapped phase distribution of the stripes through performing an algebraic operation and a stitching operation on the stripe images, and performing a spatial phase expansion on wrapped phases based on spatial sequence information of the stripes to obtain the phase distribution map of the surface of the imaging target.

In S203, a phase-coordinate relationship subsequent to a geometric calibration is obtained, and the phase distribution is converted into three-dimensional coordinates using the phase-coordinate relationship obtained subsequent to the geometric calibration.

A method for obtaining the phase-coordinate relationship is described as follows. The geometric calibration includes a camera calibration, a projector calibration, and a joint calibration, which yield an internal parameter and a distortion parameter of each of the CCD camera and the projector and a relative geometric parameter between the CCD camera and the projector. The three sets of parameters, a camera parameter, a projector parameter, and a relative geometric parameter, constitute the phase-coordinate relationship. In particular, phases correspond to projector image coordinates in a one-to-one correspondence. The projector image coordinates may be converted into three-dimensional coordinates in a projector coordinate system through the projector parameter. The three-dimensional coordinates in the projector coordinate system may be converted into three-dimensional coordinates in a CCD camera coordinate system through the relative parameter. Also, coordinates of an image captured by the CCD camera may be converted into three-dimensional coordinates in the CCD camera coordinate system through the camera parameter. Therefore, three-dimensional coordinates in the CCD camera coordinate system can be calculated with known phases and known CCD camera image coordinates, which is the phase-coordinate relationship.

In S204, an angle between an imaging support and an imaging system is adjusted, and steps S201 to S203 are repeated.

In S205, multi-angle three-dimensional coordinates of the imaging target are obtained by adjusting the angle, such that the three-dimensional surface contour image of the imaging target is obtained.

In S3, an alignment is performed on the two-dimensional bio-optical image obtained in step S1 and the three-dimensional surface contour image obtained in step S2. An alignment method is specifically described as follows. The camera parameter has been obtained in the previous stage of geometric calibration. The CCD camera image coordinates may be converted into the three-dimensional coordinates in the CCD camera coordinate system using the camera parameter. That is, each point on the CCD camera image (which refers to a two-dimensional bioluminescence image) is back-projected onto the three-dimensional surface contour using the camera parameter. The alignment between the two-dimensional bio-optical image and the three-dimensional surface contour image is therefore accomplished.

Specifically, since both the two-dimensional bio-optical image and the three-dimensional surface contour image are captured using a same CCD camera, the alignment is performed based on the CCD camera parameter.

In S4, a three-dimensional bio-optical image is reconstructed. A three-dimensional solid structure is obtained through filling the obtained three-dimensional surface contour image with biological tissues having different optical properties. A three-dimensional bioluminescence image (BLT) or a three-dimensional molecular fluorescence image (FMT) may be reconstructed in 3D through combining the three-dimensional solid structure with data obtained based on the alignment in step S2, i.e., a correspondence between points on the two-dimensional bio-optical image and points on the three-dimensional surface contour image and a corresponding optical signal intensity.

In the present disclosure, the bio-optical image and the three-dimensional surface contour image are obtained by the same CCD camera. An optical imaging operation is performed on the imaging target using each of laser light and structured light. A three-dimensional luminescence image or a three-dimensional molecular fluorescence image of the imaging target may be generated through a reconstruction subsequent to the alignment. Both optical information of molecules inside the imaging target and external three-dimensional spatial features of the imaging target can be provided.

Finally, it should be noted that the above contents are used only to explain technical solutions of the present disclosure, rather than to limit the protection scope of the present disclosure. Simple modifications or equivalent replacements can be made to the technical solutions of the present disclosure by those skilled in the art, without departing from the essence and the scope of the technical solutions of the present disclosure.

What is claimed is:

1. A three-dimensional (3D) optical imaging method, comprising the following steps:
S1, obtaining a two-dimensional bio-optical image of an imaging target by a Charge Coupled Device (CCD) camera, the two-dimensional bio-optical image being a bioluminescence image or a molecular fluorescence image;
S2, obtaining a three-dimensional surface contour image of the imaging target by using the CCD camera in step S1 in combination with structured light;
S3, performing an alignment on the two-dimensional bio-optical image obtained in step S1 and the three-dimensional surface contour image obtained in step S2; and
S4, obtaining a three-dimensional solid structure through filling the obtained three-dimensional surface contour image with biological tissues having different optical properties, and reconstructing a three-dimensional bioluminescence image or a three-dimensional molecular fluorescence image through combining the three-dimensional solid structure with data obtained based on the alignment in step S3,
wherein the two-dimensional bio-optical image of the imaging target obtained in step S1 and the three-dimensional surface contour image of the imaging target obtained in step S2 are obtained by a same CCD camera.

2. The 3D optical imaging method according to claim 1, wherein step S2 comprises the following steps:
S201, turning on a projector, projecting modulated stripe-patterned structured light onto a surface of the imaging target, and capturing stripes on the surface of the imaging target using the CCD camera;
S202, processing the stripes to obtain a phase distribution map of the surface of the imaging target;
S203, obtaining a phase-coordinate relationship subsequent to a geometric calibration, and converting the phase distribution into three-dimensional coordinates using the phase-coordinate relationship obtained subsequent to the geometric calibration;
S204, adjusting an angle between an imaging support and an imaging system, and repeating steps S201 to S203; and
S205, obtaining multi-angle three-dimensional coordinates of the imaging target by adjusting the angle, such that the three-dimensional surface contour image of the imaging target is obtained.

3. The 3D optical imaging method according to claim 2, wherein the phase distribution map of the surface of the imaging target is obtained through: obtaining stripe images of different phases contained in images captured each time, obtaining a wrapped phase distribution of the stripes through performing an algebraic operation and a stitching operation on the stripe images, and performing a spatial phase expansion on wrapped phases based on spatial sequence information of the stripes to obtain the phase distribution map of the surface of the imaging target.

4. The 3D optical imaging method according to claim 2, wherein the geometric calibration of the phase-coordinate relationship is performed by: given phases and CCD camera image coordinates that are known, converting the CCD camera image coordinates into three-dimensional coordinates in a CCD camera coordinate system using a camera parameter, converting the three-dimensional coordinates in the CCD camera coordinate system into three-dimensional coordinates in a projector coordinate system based on a relative parameter between the projector and the CCD camera, converting the three-dimensional coordinates in the projector coordinate system into projector image coordinates based on a projector parameter, and obtaining the phase-coordinate relationship based on a one-to-one correspondence between the phases and the projector image coordinates

5. The 3D optical imaging method according to claim 1, wherein the molecular fluorescence image is obtained through: turning on an excitation light source, emitting laser light by the excitation light source to irradiate the imaging target, exciting fluorescent molecules carried by an imaging object, and generating emission fluorescence; and obtaining the two-dimensional bio-optical image through collecting and processing, by the CCD camera, the generated emission fluorescence subsequent to the generated emission fluorescence being reflected by a reflection mirror and passing through a filter, or subsequent to the generated emission fluorescence passing through the filter without being reflected.

6. The 3D optical imaging method according to claim 1, wherein the bioluminescence image is obtained through: releasing a bioluminescence signal in response to a chemical reaction inside an imaging object, and obtaining a two-dimensional biological image through collecting and processing, by the CCD camera, the released bioluminescence signal subsequent to the released bioluminescence signal being reflected by a reflection mirror and passing through a filter, or obtaining the two-dimensional bio-optical image through collecting and processing, by the CCD camera, the released bioluminescence signal subsequent to the released bioluminescence signal passing through the filter without being reflected, or obtaining the two-dimensional bio-optical image through directly collecting and processing, by the CCD camera, the released bioluminescence signal without the released bioluminescence signal being reflected or passing through the filter.

7. The 3D optical imaging method according to claim 1, wherein the data obtained based on the alignment in step S3 described in step S4 comprises a correspondence between points on the two-dimensional bio-optical image and points on the three-dimensional surface contour image and a corresponding optical signal intensity.

8. A three-dimensional (3D) optical imaging system, wherein the 3D optical imaging system uses a 3D optical imaging method comprising the following steps:
S1, obtaining a two-dimensional bio-optical image of an imaging target by a Charge Coupled Device (CCD) camera, the two-dimensional bio-optical image being a bioluminescence image or a molecular fluorescence image;
S2, obtaining a three-dimensional surface contour image of the imaging target by using the CCD camera in step S1 in combination with structured light;
S3, performing an alignment on the two-dimensional bio-optical image obtained in step S1 and the three-dimensional surface contour image obtained in step S2; and
S4, obtaining a three-dimensional solid structure through filling the obtained three-dimensional surface contour image with biological tissues having different optical properties, and reconstructing a three-dimensional bioluminescence image or a three-dimensional molecular fluorescence image through combining the three-dimensional solid structure with data obtained based on the alignment in step S3, wherein the two-dimensional bio-optical image of the imaging target obtained in step S1 and the three-dimensional surface contour image of the imaging target obtained in step S2 are obtained by a same CCD camera,
wherein the 3D optical imaging system comprises the imaging support and the imaging system, the imaging system comprising the excitation light source and the CCD camera, the imaging target being fixed at the imaging support, the excitation light source and the CCD camera being disposed at a same side of the imaging target or the excitation light source and the CCD camera being disposed at two sides of the imaging target respectively, and the projector being disposed at a side of the imaging target.

9. The 3D optical imaging system according to claim 8, wherein a reflection mirror is disposed between the imaging target and the CCD camera, and the projector is disposed at a side of the reflection mirror.

10. The 3D optical imaging system according to claim 8, wherein a filter is disposed between the reflection mirror and the CCD camera.
